(19) United States
(12) Patent Application Publication
(10) Pub. No.: US 2006/0015160 A1
(43) Pub. Date: Jan. 19, 2006

(54) Method and apparatus for cooling spinal cord and fluid

(75) Inventor: Donald J. Larnard, North Hampton, NH (US)

Correspondence Address: CHRISTOPHER & WEISBERG, P.A., 200 EAST LAS OLAS BOULEVARD, SUITE 2040, FORT LAUDERDALE, FL, 33301, US

(73) Assignee: Seacoast Technologies, Inc.

(21) Appl. No.: 10/894,525

(22) Filed: Jul. 19, 2004

(51) Int. Cl. A61F 7/00; A61F 7/12

(52) U.S. Cl. 607/96; 607/105; 607/113

(57) ABSTRACT

A method and system are provided for reducing cerebrospinal tissue swelling and inflammation and providing neuroprotection. In an exemplary method, a medical procedure includes the step of subdermally, subdurally, or epidurally cooling spinal tissue. Such cooling can be effected by exposing spinal tissue, placing a cooling structure on the exposed spinal tissue, and cooling the exposed spinal tissue with the cooling structure to below body temperature for an extended period of time. Alternatively, a fluid below body temperature can be circulated through the spinal canal. Exemplary fluids include cerebrospinal fluid and non-bodily fluids such as saline.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a medical method and apparatus for treating swelling of the spinal cord and related nerve tissue and providing neuroprotection to such tissues.

### BACKGROUND OF THE INVENTION

In back injuries, neurological damage to the spinal cord, the tissue that surrounds it, and nerves that join the spinal cord can be caused by tissue swelling within the confined space of spinal canal that is defined by the vertebrae. With increased swelling, the pressure of the cerebrospinal fluid increases and spinal tissue impinges upon the hard bone of the vertebrae and where spaces exist, the injured tissue presses outward from its normal containment in the longitudinal passage defined by the spinal canal. The impingement of the soft nerve tissue against the hard bone can result in temporary or permanent nerve damage and possible paralysis or functional impairment of portions of the body or its organs.

One approach to dealing with spinal cord and associated nerve swelling, known as a laminectomy (open decompression), includes the removal of a small portion of the vertebral bone over the nerve root and/or disc material from under the nerve root to give the nerve root more space and a better healing environment.

Although the laminectomy 1 can be beneficial for certain conditions, it is believed that patients would further benefit by techniques and apparatus that reduce tissue swelling and/or reduce the duration of the inflamed condition.

### SUMMARY OF THE INVENTION

The present invention advantageously provides a method and system for reducing the extent and duration of swelling of a spinal cord and related tissues and providing neuroprotection by cooling.

In an exemplary method, a medical procedure is provided for inhibiting swelling or inflammation of spinal tissue and/or providing neuroprotection. The method includes the step of subdermally (locally, beneath the skin), epidurally, or subdurally cooling spinal tissue. Such cooling can be effected by exposing spinal tissue, placing a cooling structure on the exposed spinal tissue, and cooling the exposed spinal tissue with the cooling structure to below body temperature for an extended period of time. Alternatively, a fluid below body temperature can be circulated through the spinal canal. Exemplary fluids include cerebrospinal fluid and non-bodily fluids such as saline.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 illustrates a portion of the spine with skin and bone removed to revel the spinal canal;

FIG. 2 illustrates an exemplary cooling system used to perform a medical procedure in accordance with the present invention;

FIG. 3 illustrates a cooling structure of the system of FIG. 2 positioned within the spinal canal and in contact with spinal tissue;

FIG. 4 illustrates additional details of an exemplary cooling structure;

FIG. 5 illustrates an alternate medical procedure in accordance with the invention; and

FIG. 6 illustrates yet another medical procedure in accordance with the invention.

### DETAILED DESCRIPTION OF THE INVENTION

The present invention includes a medical procedure for inhibiting cerebrospinal tissue swelling, decreasing tissue inflammations, and reducing cerebrospinal fluid pressure by cooling tissue or cerebrospinal fluid directly and/or by cooling cerebrospinal fluid to cool tissue indirectly. In addition, cooling tissue also has the effect of providing neuroprotection to tissues as the reduction of temperature has the effect of lowering cellular metabolism and reducing chemical reactions in damaged or compromised tissue.

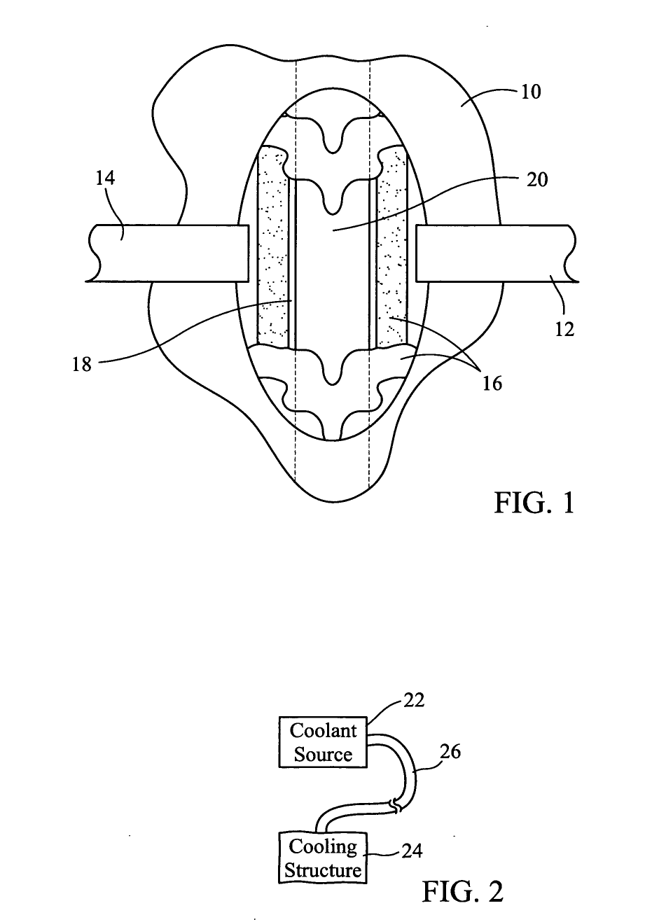

In an exemplary procedure, known medical techniques are used to retract skin and muscle and to expose and remove vertebral bone, thereby providing access to the spinal canal and spinal tissue. As shown in FIG. 1, a portion of a patient's back 10 is shown. A longitudinal incision is made and retractors 12 and 14 are used to laterally draw and hold soft tissue thereby exposing vertebral bone 16. A portion of bone is excised to expose the spinal canal 18 within which the spinal cord 20 and its protective covering tissue are located. Again, using known techniques, the dura covering the spinal cord 20 can be cut to expose the spinal cord or, alternatively, the dura is left intact.

Having determined whether the spinal cord is to be cooled directly or indirectly through the dura, a cooling structure as described below is placed directly on the exposed tissue. The cooling structure is then cooled to a predetermined temperature below normal body temperature (98.6 degrees F.), but above freezing (32 degrees F.), thereby cooling the tissue to a temperature above freezing, but below body temperature. The cooling structure is left in place on the tissue for a selected period of time or until tissue swelling has subsided. To the extent possible, the cooling structure is dimensioned so as to cover and in be in contact with substantially all of the exposed tissue.

Turning now to FIG. 2, an exemplary system is illustrated that is suitable for performing the procedure in accordance with the invention. The system includes a source of coolant 22, a cooling structure 24, and an umbilical 26 connecting the source of coolant and the cooling structure and conducting cooling fluid therebetween. The terms “coolant” and “cooling fluid” as used interchangeably herein generally refer to any flowable substance, including but not limited to gasses, liquids, and combinations thereof. In an exemplary system, the fluid is a saline solution.

Although the source of coolant 22 can be a reservoir from which cooling fluid is expelled to be collected elsewhere or discarded, in view of the expected long duration of the above described treatment, a looped or closed system is believed to be desirable, wherein the cooling fluid is continuously recirculated between the source of coolant and the cooling structure. More particularly, the source of coolant 22 can include a heat exchanger, a pump, and a control mechanism. Temperature, pressure and flow sensors are distributed through the system to provide information to the control mechanism which can, in response to sensor data, alter the performance of the system. The control mechanism can be set or programmed to maintain a specific temperature of the cooling structure and/or to monitor and maintain tissue at a selected temperature.

The cooling structure 24 can include a very flexible element, having a surface area that readily follows the curvature of the tissue surface, through which the cooling fluid is circulated. Although the cooling structure 24 can be circular, square, rectangular, or irregular, the particular shape is not significant. Rather, the feature of importance is that the cooling structure 24 covers as much exposed tissue as possible and readily conforms to the tissue without injuring it or causing further trauma. Exemplary materials suitable for the cooling structure 24 include rubber, silicone, flexible polymers and other bio-compatible materials known in the art.

In an exemplary embodiment, as shown in FIG. 4, the cooling structure 24 includes a serpentine channel 30 through which cooling fluid travels. The cooling fluid enters and exits the cooling structure 24 through the umbilical 26.

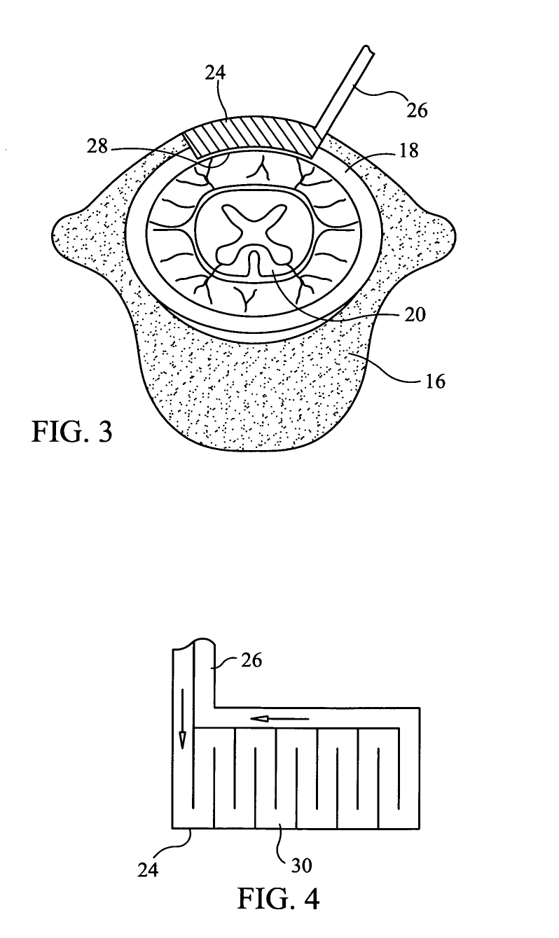

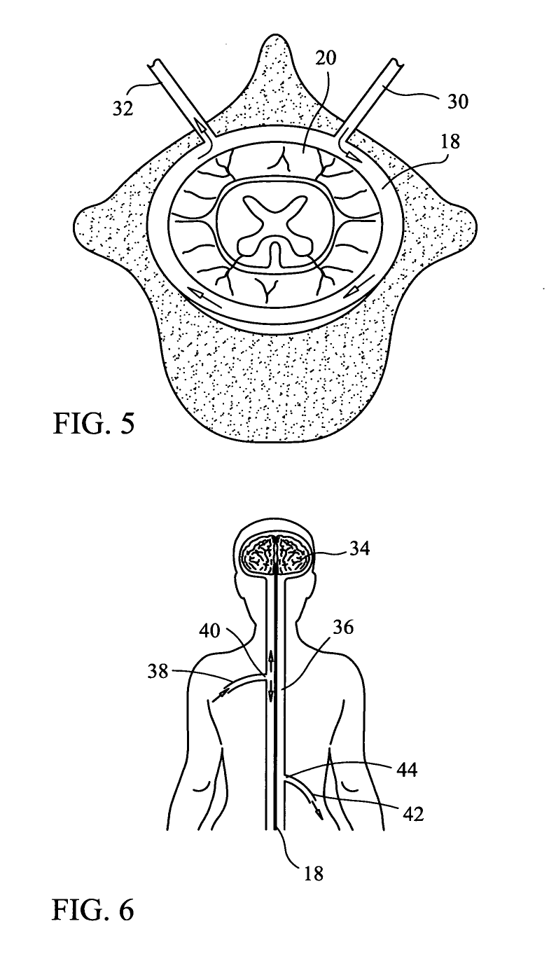

Referring now to FIG. 3, a transverse sectional view of vertebral bone 16, the spinal canal 18, and the spinal cord 20 are shown. The cooling structure 24 is also shown in sectional view, wherein it is located in the gap created by cutting away vertebral bone. As shown, the cooling structure 24 is disposed on the surface 28 of the spinal tissue, which in this example is the dura.

Whereas the medical procedure illustrated in FIG. 3 is advantageous for localized tissue cooling, reference is now made to FIGS. 5 and 6 which are directed to indirect tissue cooling and cooling by fluid perfusion. For example, FIG. 5 illustrates a transverse sectional view of vertebral bone 16, the spinal canal 18, and the spinal cord 20. Relatively small openings (about 1 cm in diameter or less) are created in the vertebral bone to provide access to the spinal canal. A first conduit 30 conducts coolant from a coolant source through one of the openings in the bone so that coolant flows into the spinal canal 18 and circulates therethrough as represented by the arrows. Coolant exits the spinal canal 18 through the other opening and an associated second conduit 32.

In an alternative procedure to that illustrated in FIG. 5, spinal fluid is withdrawn from the spinal canal 18 from one of the openings, cooled outside of the body, and reintroduced while cool back into the body through another opening.

Although the cooling effect described above with respect to FIG. 5 can extend beyond the site of fluid introduction, FIG. 6 illustrates a technique for enlarging the scale of cooling so as to affect more tissue. For example, a representation of a spinal cord 18 and brain 34, surrounded by tissue that contains same and defines a path 36 for flow of cerebrospinal fluid, is shown. Chilled coolant or cerebrospinal fluid is introduced through a first conduit 38 at a first point 40 into the path 36 and removed from the path through a second conduit 42 at a second point longitudinally separated from the first point. Thus, the chilled coolant or cerebrospinal fluid can cool tissue along the spine or around the brain.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A medical procedure comprising the steps of:
subdermally cooling spinal tissue.

2. The medical procedure of claim 1, wherein the subdermal cooling is effected epidurally.

3. The medical procedure of claim 1, wherein the subdermal cooling is effected subdurally.

4. The medical procedure of claim 1, wherein the step subdermally cooling spinal tissue comprises the steps of:
exposing spinal tissue;
placing a cooling structure on the exposed spinal tissue; and
cooling the exposed spinal tissue with the cooling structure.

5. The medical procedure of claim 4, wherein the step of placing a cooling structure on the exposed spinal tissue includes the step of placing the cooling structure over and in contact with substantially all of the exposed spinal tissue.

6. The medical procedure of claim 5, wherein the step of cooling the exposed spinal tissue includes the step of cooling a tissue contacting surface of the cooling structure to a temperature in the range of 98 degrees F. to 33 degrees F. for a period of time greater than one day.

7. The medical procedure of claim 6, wherein the period of time greater than one day is a period of time greater than one week.

8. The medical procedure of claim 1, wherein the step of subdermally cooling spinal tissue comprises the step of:
circulating a fluid below body temperature through the spinal canal.

9. A medical procedure comprising the step of:
cooling cerebrospinal fluid.

10. The medical procedure of claim 9, wherein the cerebrospinal fluid is cooled while it is within the body.

11. The medical procedure of claim 9, further comprising the steps of:
routing cerebrospinal fluid outside the body;
cooling the cerebrospinal fluid while it is outside the body to a temperature below body temperature; and
reintroducing the cerebrospinal fluid into the body.

12. A medical procedure comprising the step of:
circulating a fluid below body temperature through the spinal canal.

13. The medical procedure of claim 12, wherein the fluid is contained by a cooling structure.

14. The medical procedure of claim 12, wherein the fluid is contained by the body.

15. The medical procedure of claim 12, wherein the fluid is cerebrospinal fluid.

16. The medical procedure of claim 12, wherein the fluid is a non-bodily fluid.

17. The medical procedure of claim 16, wherein the fluid is a saline solution.

18. The medical procedure of claim 12, further comprising the steps of introducing the fluid at a first longitudinal point along the spine and withdrawing the fluid at a second longitudinal point along the spine.

19. The medical procedure of claim 12, further comprising the steps of introducing the fluid at a first lateral point along the spine and withdrawing the fluid at a second lateral point along the spine.

20. The medical procedure of claim 12, wherein the first and second lateral points are at substantially the same longitudinal location along the spine.
